(19) United States
(12) Patent Application Publication
(10) Pub. No.: US 2026/0162783 A1
(43) Pub. Date: Jun. 11, 2026

(54) METHOD FOR MEDICAL RECORD DATA EXTRACTION AND SUMMARIZATION USING LARGE LANGUAGE ARTIFICIAL INTELLIGENCE MODELS

(71) Applicant: Mitchell International, Inc., San Diego, CA (US)

(72) Inventors: Michael Cwynar, San Diego, CA (US); Uyen Hoang, San Diego, CA (US); Norman Tyrrell, San Diego, CA (US)

(21) Appl. No.: 18/971,246

(22) Filed: Dec. 6, 2024

(51) Int. Cl. G16H 10/60; G06F 40/40; G06V 30/416

(52) U.S. Cl. CPC G16H 10/60; G06F 40/40; G06V 30/416

(57) ABSTRACT

Embodiments of the disclosure provide systems and methods for using large language artificial intelligence models to extract and summarize data from medical records. Extracting and summarizing unstructured data from medical records can comprise receiving a set of documents comprising medical records. The received set of documents can be pre-processed into a set of medical information. A Large Language Model (LLM) can then be queried for summary information of the set of relevant medical information. The summary information can then be provided to one or more workflows for further processing.

### FIELD OF THE DISCLOSURE

Embodiments of the present disclosure relate generally to methods and systems for processing of medical records and more particularly to using large language artificial intelligence models to extract and summarize data from medical records.

### BACKGROUND

Medical records can be a rich source of critical data in evaluating medical claims but are often overlooked in the review process since they can be lengthy and disorganized.

Existing methods and practices for evaluating medical claims based on medical records suffer from two limitations. Purely manual, human-driven processes require trained users to read, organize, and summarize all of the information in the original documents. This approach is slow and labor-intensive resulting in a delay in the service becoming available and a high cost. Automated methods utilizing traditional Natural Language Processing (NLP) systems to extract information from medical records often return too much information and still require manual review of the extracted information. Hence, there is a need for improved methods and systems for processing of medical records.

### BRIEF SUMMARY

Embodiments of the disclosure provide systems and methods for using large language artificial intelligence models to extract and summarize data from medical records. According to one embodiment, a method for extracting and summarizing unstructured data from medical records can comprise receiving a set of documents comprising medical records. The received set of documents can be pre-processed into a set of medical information. Pre-processing the received set of documents into a set of medical information can comprise sorting and classifying the received set of documents. Pre-processing the received set of documents into a set of medical information can further comprise running an Optical Character Recognition (OCR) process on the received set of documents.

A Large Language Model (LLM) can then be queried for summary information of the set of relevant medical information. Querying the LLM for summary information of the set of medical information can comprise providing, to the LLM, a sequence of successively more specific queries related to the set of medical information and summary information returned by the LLM. In some cases, the LLM can be queried for a summary of the summary information.

The summary information can then be provided to one or more workflows for further processing. For example, providing the summary information to one or more workflows for further processing can comprise providing the summary information to a review and processing tool. The review and processing tool can provide a user interface through which a user interacts with the summary information. The user interface provided by the review and processing tool can present the summary information organized into a plurality of groups and provide an indication of specific information within the summary information requiring review. A modification of the summary information can be received from the review tool based on an input from the user through the user interface and the summary information can be updated based on the received modification. In another example, providing the summary information to one or more workflows for further processing can additionally, or alternatively, comprise providing the summary information to a report generation process.

According to another embodiment, a system can comprise a processor and a memory coupled with and readable by the processor. The memory can store therein a set of instructions which, when executed by the processor, causes the processor to receive a set of documents comprising medical records and pre-process the received set of documents into a set of medical information. Pre-processing the received set of documents into a set of medical information can comprise sorting and classifying the received set of documents. Pre-processing the received set of documents into a set of medical information can further comprise running an OCR process on the received set of documents.

The instructions can further cause the processor to query an LLM for summary information of the set of relevant medical information. Querying the LLM for summary information of the set of medical information can comprise providing, to the LLM, a sequence of successively more specific queries related to the set of medical information and summary information returned by the LLM. In some cases, the set of instructions can further cause the processor to query the LLM for a summary of the summary information.

The instructions can further cause the processor to provide the summary information to one or more workflows for further processing. Providing the summary information to one or more workflows for further processing can comprise, for example, providing the summary information to a review and processing tool. The review and processing tool can provide a user interface through which a user interacts with the summary information. The user interface provided by the review and processing tool can present the summary information organized into a plurality of groups and provide an indication of specific information within the summary information requiring review. A modification of the summary information can be received from the review tool based on an input from the user through the user interface and the summary information can be updated based on the received modification. In such cases, the set of instructions can further cause the processor to receive a modification of the summary information from the review tool and update the summary information based on the received modification. Additionally, or alternatively, providing the summary information to one or more workflows for further processing can comprise providing the summary information to a report generation process.

According to yet another embodiment, a non-transitory, computer-readable medium can comprise a set of instructions stored therein which, when executed by a processor, causes the processor to receive a set of documents comprising medical records and pre-process the received set of documents into a set of medical information. Pre-processing the received set of documents into a set of medical information can comprise sorting and classifying the received set of documents. Pre-processing the received set of documents into a set of medical information can further comprise running an OCR process on the received set of documents.

The instructions can further cause the processor to query an LLM for summary information of the set of relevant medical information. Querying the LLM for summary information of the set of medical information can comprise providing, to the LLM, a sequence of successively more specific queries related to the set of medical information and summary information returned by the LLM. In some cases, the set of instructions can further cause the processor to query the LLM for a summary of the summary information.

The instructions can further cause the processor to provide the summary information to one or more workflows for further processing. Providing the summary information to one or more workflows for further processing can comprise, for example, providing the summary information to a review tool. In such cases, the set of instructions can further cause the processor to receive a modification of the summary information from the review tool and update the summary information based on the received modification. Additionally, or alternatively, providing the summary information to one or more workflows for further processing can comprise providing the summary information to a report generation process.

### BRIEF DESCRIPTION OF THE DRAWINGS

FIG. 1 is a block diagram illustrating elements of an exemplary computing environment in which embodiments of the present disclosure may be implemented.

FIG. 2 is a block diagram illustrating elements of an exemplary computing device in which embodiments of the present disclosure may be implemented.

FIG. 3 is a block diagram illustrating an exemplary environment for extracting and summarizing unstructured data from medical records according to one embodiment of the present disclosure.

FIG. 4 is a block diagram illustrating stages of an exemplary workflow for extracting and summarizing unstructured data from medical records according to one embodiment of the present disclosure.

FIG. 5 is a flowchart illustrating an exemplary process for extracting and summarizing unstructured data from medical records according to one embodiment of the present disclosure.

FIG. 6 is a flowchart illustrating additional details of an exemplary process for pre-processing of medical records according to one embodiment of the present disclosure.

FIG. 7 is a flowchart illustrating additional details of an exemplary process for querying an LLM according to one embodiment of the present disclosure.

FIG. 8 is a flowchart illustrating additional details of an exemplary process for providing summary information to one or more workflows for further processing according to one embodiment of the present disclosure.

In the appended figures, similar components and/or features may have the same reference label. Further, various components of the same type may be distinguished by following the reference label by a letter that distinguishes among the similar components. If only the first reference label is used in the specification, the description is applicable to any one of the similar components having the same first reference label irrespective of the second reference label.

### DETAILED DESCRIPTION

In the following description, for the purposes of explanation, numerous specific details are set forth in order to provide a thorough understanding of various embodiments disclosed herein. It will be apparent, however, to one skilled in the art that various embodiments of the present disclosure may be practiced without some of these specific details. The ensuing description provides exemplary embodiments only and is not intended to limit the scope or applicability of the disclosure. Furthermore, to avoid unnecessarily obscuring the present disclosure, the preceding description omits a number of known structures and devices. This omission is not to be construed as a limitation of the scopes of the claims. Rather, the ensuing description of the exemplary embodiments will provide those skilled in the art with an enabling description for implementing an exemplary embodiment. It should however be appreciated that the present disclosure may be practiced in a variety of ways beyond the specific detail set forth herein.

While the exemplary aspects, embodiments, and/or configurations illustrated herein show the various components of the system collocated, certain components of the system can be located remotely, at distant portions of a distributed network, such as a Local-Area Network (LAN) and/or Wide-Area Network (WAN) such as the Internet, or within a dedicated system. Thus, it should be appreciated, that the components of the system can be combined in to one or more devices or collocated on a particular node of a distributed network, such as an analog and/or digital telecommunications network, a packet-switch network, or a circuit-switched network. It will be appreciated from the following description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire and fiber optics, and may take the form of acoustic or light waves, such as those generated during radio-wave and infra-red data communications.

As used herein, the phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The term “computer-readable medium” as used herein refers to any tangible storage and/or transmission medium that participate in providing instructions to a processor for execution. Such a medium may take many forms, including but not limited to, non-volatile media, volatile media, and transmission media. Non-volatile media includes, for example, Non-Volatile Random-Access Memory (NVRAM), or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a Compact Disk Read-Only Memory (CD-ROM), any other optical medium, punch cards, paper tape, any other physical medium with patterns of holes, a Random-Access Memory (RAM), a Programmable Read-Only Memory (PROM), and Erasable Programable Read-Only Memory (EPROM), a Flash-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

A “computer readable signal” medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable medium may be transmitted using any appropriate medium, including but not limited to wireless, wireline, optical fiber cable, Radio Frequency (RF), etc., or any suitable combination of the foregoing.

The terms “determine,” “calculate,” and “compute,” and variations thereof, as used herein, are used interchangeably and include any type of methodology, process, mathematical operation or technique.

It shall be understood that the term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary of the disclosure, brief description of the drawings, detailed description, abstract, and claims themselves.

Aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Any combination of one or more computer readable medium(s) may be utilized. The computer readable medium may be a computer readable signal medium or a computer readable storage medium.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as Programmable Logic Device (PLD), Programmable Logic Array (PLA), Field Programmable Gate Array (FPGA), Programmable Array Logic (PAL), special purpose computer, any comparable means, or the like. In general, any device(s) or means capable of implementing the methodology illustrated herein can be used to implement the various aspects of this disclosure. Exemplary hardware that can be used for the disclosed embodiments, configurations, and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

Examples of the processors as described herein may include, but are not limited to, at least one of Qualcomm® Snapdragon® 800 and 801, Qualcomm® Snapdragon® 610 and 615 with 4G LTE Integration and 64-bit computing, Apple® A 7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or Very Large-Scale Integration (VLSI) design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or Common Gateway Interface (CGI) script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

Various additional details of embodiments of the present disclosure will be described below with reference to the figures. While the flowcharts will be discussed and illustrated in relation to a particular sequence of events, it should be appreciated that changes, additions, and omissions to this sequence can occur without materially affecting the operation of the disclosed embodiments, configuration, and aspects.

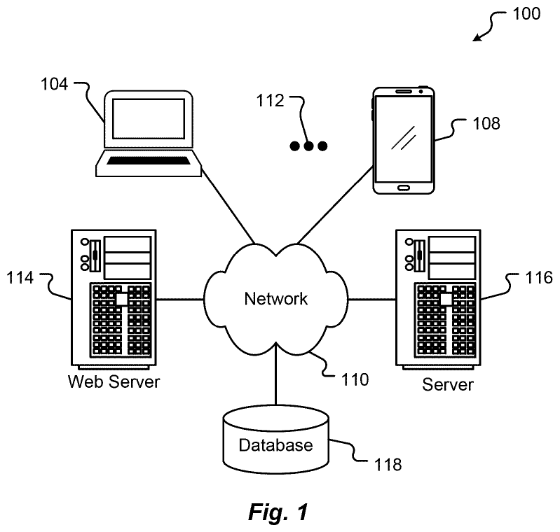

FIG. 1 is a block diagram illustrating elements of an exemplary computing environment in which embodiments of the present disclosure may be implemented. More specifically, this example illustrates a computing environment 100 that may function as the servers, user computers, or other systems provided and described herein. The environment 100 includes one or more user computers, or computing devices, such as a computing device 104, a communication device 108, and/or more 112. The computing devices 104, 108, 112 may include general purpose personal computers (including, merely by way of example, personal computers, and/or laptop computers running various versions of Microsoft Corp.'s Windows® and/or Apple Corp.'s Macintosh® operating systems) and/or workstation computers running any of a variety of commercially-available UNIX® or UNIX-like operating systems. These computing devices 104, 108, 112 may also have any of a variety of applications, including for example, database client and/or server applications, and web browser applications. Alternatively, the computing devices 104, 108, 112 may be any other electronic device, such as a thin-client computer, Internet-enabled mobile telephone, and/or personal digital assistant, capable of communicating via a network 110 and/or displaying and navigating web pages or other types of electronic documents. Although the exemplary computer environment 100 is shown with two computing devices, any number of user computers or computing devices may be supported.

Environment 100 further includes a network 110. The network 110 may can be any type of network familiar to those skilled in the art that can support data communications using any of a variety of commercially-available protocols, including without limitation Session Initiation Protocol (SIP), Transmission Control Protocol/Internet Protocol (TCP/IP), Systems Network Architecture (SNA), Internetwork Packet Exchange (IPX), AppleTalk, and the like. Merely by way of example, the network 110 maybe a Local Area Network (LAN), such as an Ethernet network, a Token-Ring network and/or the like; a wide-area network; a virtual network, including without limitation a Virtual Private Network (VPN); the Internet; an intranet; an extranet; a Public Switched Telephone Network (PSTN); an infra-red network; a wireless network (e.g., a network operating under any of the IEEE 802.9 suite of protocols, the Bluetooth® protocol known in the art, and/or any other wireless protocol); and/or any combination of these and/or other networks.

The system may also include one or more servers 114, 116. In this example, server 114 is shown as a web server and server 116 is shown as an application server. The web server 114, which may be used to process requests for web pages or other electronic documents from computing devices 104, 108, 112. The web server 114 can be running an operating system including any of those discussed above, as well as any commercially-available server operating systems. The web server 114 can also run a variety of server applications, including SIP servers, HyperText Transfer Protocol (secure) (HTTP(s)) servers, FTP servers, CGI servers, database servers, Java servers, and the like. In some instances, the web server 114 may publish operations available operations as one or more web services.

The environment 100 may also include one or more file and or/application servers 116, which can, in addition to an operating system, include one or more applications accessible by a client running on one or more of the computing devices 104, 108, 112. The server(s) 116 and/or 114 may be one or more general purpose computers capable of executing programs or scripts in response to the computing devices 104, 108, 112. As one example, the server 116, 114 may execute one or more web applications. The web application may be implemented as one or more scripts or programs written in any programming language, such as JavaTM, C, C#®, or C++, and/or any scripting language, such as Perl, Python, or Tool Command Language (TCL), as well as combinations of any programming/scripting languages. The application server(s) 116 may also include database servers, including without limitation those commercially available from Oracle®, Microsoft®, Sybase®, IBM® and the like, which can process requests from database clients running on a computing device 104, 108, 112.

The web pages created by the server 114 and/or 116 may be forwarded to a computing device 104, 108, 112 via a web (file) server 114, 116. Similarly, the web server 114 may be able to receive web page requests, web services invocations, and/or input data from a computing device 104, 108, 112 (e.g., a user computer, etc.) and can forward the web page requests and/or input data to the web (application) server 116. In further embodiments, the server 116 may function as a file server. Although for ease of description, FIG. 1 illustrates a separate web server 114 and file/application server 116, those skilled in the art will recognize that the functions described with respect to servers 114, 116 may be performed by a single server and/or a plurality of specialized servers, depending on implementation-specific needs and parameters. The computer systems 104, 108, 112, web (file) server 114 and/or web (application) server 116 may function as the system, devices, or components described herein.

The environment 100 may also include a database 118. The database 118 may reside in a variety of locations. By way of example, database 118 may reside on a storage medium local to (and/or resident in) one or more of the computers 104, 108, 112, 114, 116. Alternatively, it may be remote from any or all of the computers 104, 108, 112, 114, 116, and in communication (e.g., via the network 110) with one or more of these. The database 118 may reside in a Storage-Area Network (SAN) familiar to those skilled in the art. Similarly, any necessary files for performing the functions attributed to the computers 104, 108, 112, 114, 116 may be stored locally on the respective computer and/or remotely, as appropriate. The database 118 may be a relational database, such as Oracle 20i®, that is adapted to store, update, and retrieve data in response to Structured Query Language (SQL) formatted commands.

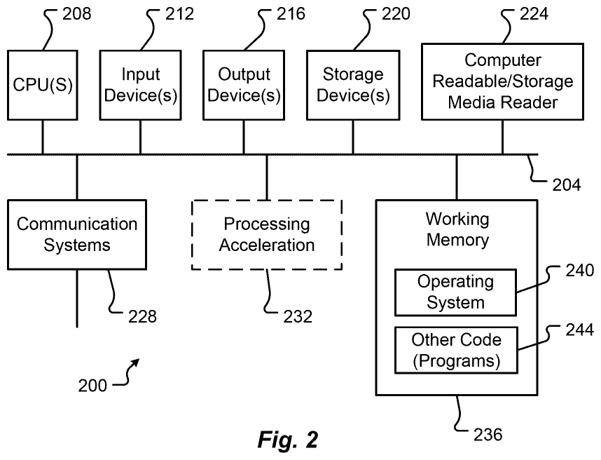

FIG. 2 is a block diagram illustrating elements of an exemplary computing device in which embodiments of the present disclosure may be implemented. More specifically, this example illustrates one embodiment of a computer system 200 upon which the servers, user computers, computing devices, or other systems or components described above may be deployed or executed. The computer system 200 is shown comprising hardware elements that may be electrically coupled via a bus 204. The hardware elements may include one or more Central Processing Units (CPUs) 208; one or more input devices 212 (e.g., a mouse, a keyboard, etc.); and one or more output devices 216 (e.g., a display device, a printer, etc.). The computer system 200 may also include one or more storage devices 220. By way of example, storage device(s) 220 may be disk drives, optical storage devices, solid-state storage devices such as a Random-Access Memory (RAM) and/or a Read-Only Memory (ROM), which can be programmable, flash-updateable and/or the like.

The computer system 200 may additionally include a computer-readable storage media reader 224; a communications system 228 (e.g., a modem, a network card (wireless or wired), an infra-red communication device, etc.); and working memory 236, which may include RAM and ROM devices as described above. The computer system 200 may also include a processing acceleration unit 232, which can include a Digital Signal Processor (DSP), a special-purpose processor, and/or the like.

The computer-readable storage media reader 224 can further be connected to a computer-readable storage medium, together (and, optionally, in combination with storage device(s) 220) comprehensively representing remote, local, fixed, and/or removable storage devices plus storage media for temporarily and/or more permanently containing computer-readable information. The communications system 228 may permit data to be exchanged with a network and/or any other computer described above with respect to the computer environments described herein. Moreover, as disclosed herein, the term “storage medium” may represent one or more devices for storing data, including ROM, RAM, magnetic RAM, core memory, magnetic disk storage mediums, optical storage mediums, flash memory devices and/or other machine-readable mediums for storing information.

The computer system 200 may also comprise software elements, shown as being currently located within a working memory 236, including an operating system 240 and/or other code 244. It should be appreciated that alternate embodiments of a computer system 200 may have numerous variations from that described above. For example, customized hardware might also be used and/or particular elements might be implemented in hardware, software (including portable software, such as applets), or both. Further, connection to other computing devices such as network input/output devices may be employed.

Examples of the processors 208 as described herein may include, but are not limited to, at least one of Qualcomm® Snapdragon® 800 and 801, Qualcomm® Snapdragon® 620 and 615 with 4G LTE Integration and 64-bit computing, Apple® A 7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

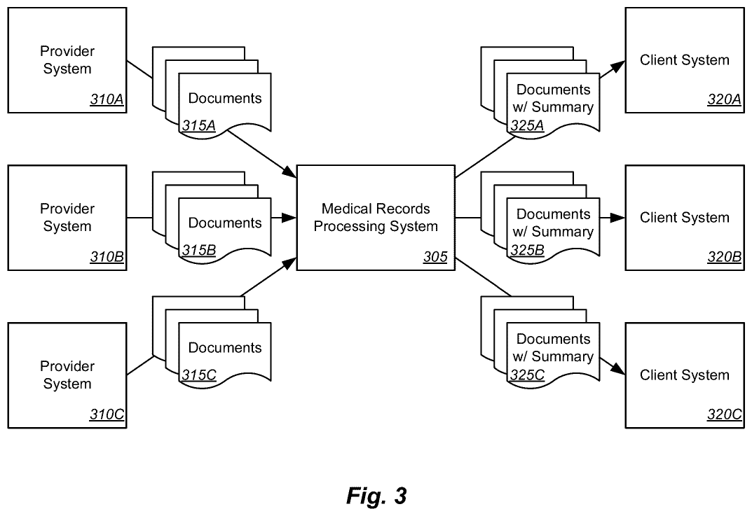

FIG. 3 is a block diagram illustrating an exemplary environment for extracting and summarizing unstructured data from medical records according to one embodiment of the present disclosure. As illustrated in this example, the environment can include a medical records processing system 305 coupled with a communications network (not shown here) The medical records processing system 305 can comprise any one or more servers and/or other computing devices as described above. The communications network can comprise any one or more wired and/or wireless, local-area and/or wide-area networks as known in the art including, but not limited to, the Internet.

Any number of provider systems 310A-310C can be in communication with the medical records processing system 305 via the communications network. The provider systems 310A-310C can each comprise any one or more servers and/or other computing devices as described above. The medical records processing system 305 can also be in communication with any number of client systems 320A-320C through the communication network. The client systems can also each comprise any one or more servers and/or other computing devices as described above.

Generally speaking, the provider systems 310A-310C can be systems of medical service providers such as hospitals, urgent-care providers, long-term care providers, doctors'offices, etc. As known in the art, the provider systems can maintain a wide range of documents 315A-315C related to patience serviced by the medical service providers. These documents 315A-315C can be submitted by the provider systems 310A-310C to the medical records processing system 305.

Once the documents 315A-315C have been submitted, the medical records processing system 305 can review these documents 315A-315C on behalf of any number of clients. The client can include, for example, third-party payors such as medical insurance companies, Medicare or Medicaid, etc. According to one embodiment and as will be described in greater detail below, the medical records processing system can use one or more Large Language Models (LLMs) to extract and summarize data from medical records documents 315A-315C. The documents and summaries 325A-325C can then be provided via the communications network to the appropriate client systems 320A-320C.

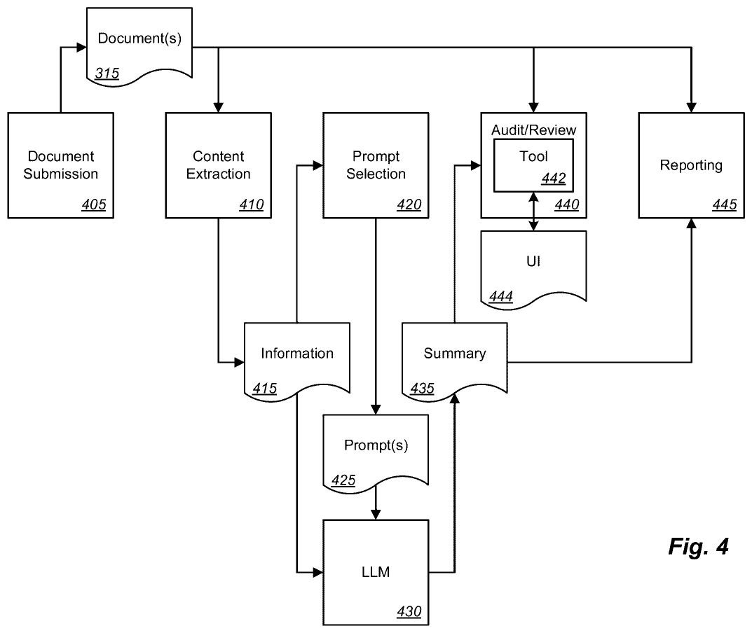

FIG. 4 is a block diagram illustrating stages of an exemplary workflow for extracting and summarizing unstructured data from medical records according to one embodiment of the present disclosure. As illustrated in this example, a workflow as may be executed by the medical records processing system 305 can begin with a document submission process 405 through which service provider systems 310A-310C can upload or otherwise submit documents 315.

A content extraction process 410 can pre-process the unstructured data of the documents 315 into a set of medical information 415 by sorting and categorizing the received set of documents. Sorting of the documents can be based on any of a number of criteria including, but not limited to document type, date, diagnostic and/or other codes within the documents, etc. The sorted documents can then be categorized or classified based on a set of rules applied to metadata and/or content of the document. Depending upon the format and/or type of document, pre-processing the received set of documents into a set of medical information can further comprise running an OCR process on the received set of documents.

A prompt selection process 420 can then iteratively select a sequence of successively more specific prompts related to the extracted medical information 415 to be applied by an LLM 430. Example output request to the LLM 430 can be “Output a list of object in json, where each object is a summary of a visit, at maximum 100 words, with the more following items: ‘Date of Service’: service date, ‘Provider’: provider or physician name that provided the medical service for the service date, ‘Complaints’: Complaints from the patient, include any stated symptoms and pain or discomfort level, ‘Exam’: Findings and assessment by the medical provider, ‘Diagnosis’: diagnosis and interpretation of testing results, ‘Plan’: Treatment plan recommended by the medical provider for the diagnosed condition, If no information is found for an item, leave blank.” Note that the utilization of a JSON format is not required but may be used to facilitate later analysis and processing downstream.

A multi-step query method can be subsequently used to further summarize the information returned from the LLM 430 and can be further broken down for this application using the following approach. Note that these can either be conducted in parallel or in series depending on the available processing power and workflow needs. A subsequent multi-step summary method can be illustrated as follows and can be requested of the LLM in a variety of different query types for the purpose of obtaining a concise summary of the medical records without extraneous details: “Given a list of summaries of medical visits, produce a list of all differences, clearly marked by date, in Complaints from one visit date to the next.” This same method can be repeated for Diagnosis, Treatment Plan, and/or for any category of information within the medical records and can include information not initially returned at the first stage.

Once the summary 435 has been generated in this way, the summary 435 can be provided to one or more additional workflows or processes. For example, the additional processes can include an audit and/or review process 440 in which revisions to the summary 435 can be submitted and the summary 435 updated accordingly. More specifically, the review process 440 can include executing a review and processing tool 442. The review and processing tool 442 can provide a user interface 444 through which a user interacts with the summary information 435. The user interface 444 provided by the review and processing tool 442 can present the summary information organized into a plurality of groups. For example, the groups can include, but are not limited to, medical insurance claim information, demographic information for the patient or claimant, medical history information for the patient or claimant, treatment or medical care information, etc. Additionally, or alternatively, the review and processing tool 442 can analyze the summary information 435 and provide through the user interface 444 an indication of specific information within the summary information 435 specifically requiring review, e.g., that is complete, was found by the review and processing tool 442 to be outside of expected information based on historical information and/or models maintained and/or accessed by the review and processing tool, etc. A modification of the summary information can be received from the review and processing tool 442 based on an input from the user through the user interface 444 and the summary information 435 can be updated based on the received modification.

Additionally, or alternatively, the process can include a reporting 445 process 445. In some cases, the reporting process can apply client specific formatting to the summary 435 and/or information therein. For example, one client may specify certain information from the summary to be provided but other information excluded. The client specific formatting may also specify a manner, e.g., textual, graphical, and or other formatting, in which information from the summary is presented.

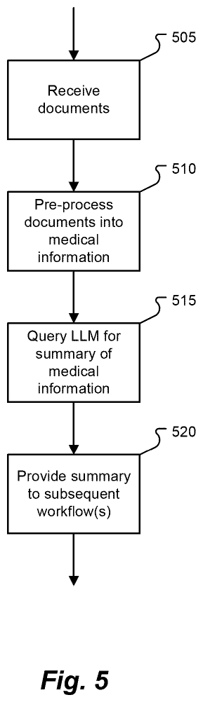

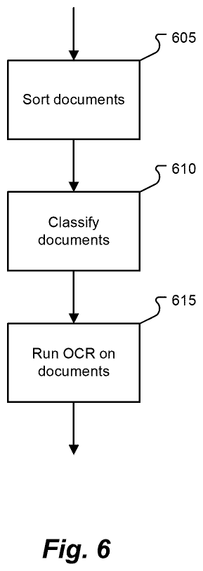

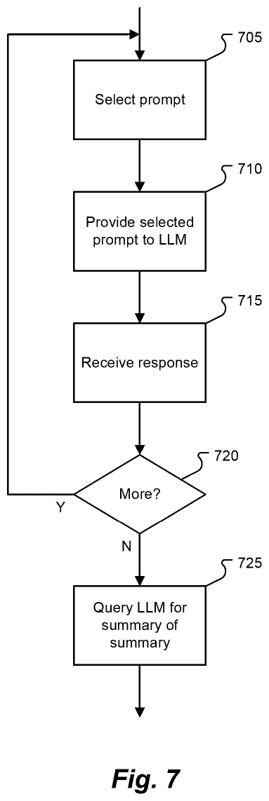

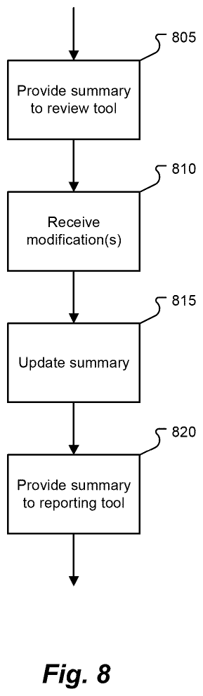

FIG. 5 is a flowchart illustrating an exemplary process for extracting and summarizing unstructured data from medical records according to one embodiment of the present disclosure. As illustrated in this example, extracting and summarizing unstructured data from medical records can comprise receiving 505 a set of documents comprising medical records. The received set of documents can be pre-processed 510 into a set of medical information. Additional details of an exemplary process for pre-processing documents into medical information will be described below with reference to FIG. 6. An LLM can then be queried for summary information of the set of relevant medical information. Additional details of an exemplary process for querying an LLM will be described below with reference to FIG. 7. The summary information can then be provided to one or more workflows for further processing. Additional details of an exemplary process for providing summary information to a set of workflows will be described below with reference to FIG. 8.

FIG. 6 is a flowchart illustrating additional details of an exemplary process for pre-processing of medical records according to one embodiment of the present disclosure. As illustrated in this example, pre-processing a set of documents into a set of medical information can comprise sorting 605 the received set of documents. Sorting 605 of the documents can be based on any of a number of criteria including, but not limited to document type, date, diagnostic and/or other codes within the documents, etc. The sorted documents can then be categorized 610 or classified based on a set of rules applied to metadata and/or content of the document. Depending upon the format and/or type of document, pre-processing the received set of documents into a set of medical information can further comprise running 615 an OCR process on the received set of documents.

FIG. 7 is a flowchart illustrating additional details of an exemplary process for querying an LLM according to one embodiment of the present disclosure. As illustrated in this example, querying an LLM for summary information of the set of medical information can comprise selecting 705 a prompt based on the pre-processed medical information. The selected 705 prompt can be provided 710 to the LLM and a set of results from the medical information can be received 715. A determination 720 can then be made as to whether to further prompt the LLM. In response to determining 720 to further prompt the LLM an additional, more specific prompt can be selected 705, the additional, more specific prompt can be provided 710 to the LLM, and a subsequent set of results from the medical information and previous results can be received 715. In this way, a sequence of successively more specific queries related to the set of medical information and previous results can be returned by the LLM. In some cases, after determining 720 no further prompts are needed related to the medical information, the LLM can be queried 725 or prompted for a summary of the summary information.

FIG. 8 is a flowchart illustrating additional details of an exemplary process for providing summary information to one or more workflows for further processing according to one embodiment of the present disclosure. As illustrated in this example, providing the summary information to one or more workflows for further processing can comprise optionally providing 805 the summary information to a providing the summary information to a review and processing tool. The review and processing tool can provide a user interface through which a user interacts with the summary information. The user interface provided by the review and processing tool can present the summary information organized into a plurality of groups and provide an indication of specific information within the summary information requiring review, e.g., that is complete, was found by the review and processing tool to be outside of expected information based on historical information and/or models maintained and/or accessed by the review and processing tool, etc. A modification of the summary information can be received 810 from the review tool based on an input from the user through the user interface and the summary information can be updated 815 based on the received modification. Providing the summary information to one or more workflows for further processing can additionally, or alternatively, comprise providing 820 the summary information to a report generation process.

The present disclosure, in various aspects, embodiments, and/or configurations, includes components, methods, processes, systems, and/or apparatus substantially as depicted and described herein, including various aspects, embodiments, configurations embodiments, sub-combinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for extracting and summarizing unstructured data from medical records, the method comprising:
receiving, by a processor of a medical records processing system, a set of documents comprising medical records;
pre-processing, by a processor of the medical records processing system, the received set of documents into a set of medical information;
querying, by a processor of the medical records processing system, a Large Language Model (LLM) for summary information of the set of relevant medical information; and
providing, by a processor of the medical records processing system, the summary information to one or more workflows for further processing.

2. The method of claim 1, wherein pre-processing the received set of documents into a set of medical information comprises sorting and classifying the received set of documents.

3. The method of claim 1, wherein pre-processing the received set of documents into a set of medical information comprises running an Optical Character Recognition (OCR) process on the received set of documents.

4. The method of claim 1, wherein querying the LLM for summary information of the set of medical information comprises providing, to the LLM, a sequence of successively more specific queries related to the set of medical information and summary information returned by the LLM.

5. The method of claim 1, wherein providing the summary information to one or more workflows for further processing comprises providing the summary information to a review and processing tool and wherein the review and processing tool provides a user interface through which a user interacts with the summary information.

6. The method of claim 5, wherein the user interface provided by the review and processing tool presents the summary information organized into a plurality of groups and provides an indication of specific information within the summary information requiring review.

7. The method of claim 5, further comprising:
receiving, by the processor of the medical records processing system, a modification of the summary information from the review and processing tool based on an input from the user through the user interface; and
updating, by the processor of the medical records processing system, the summary information based on the received modification.

8. The method of claim 1, wherein providing the summary information to one or more workflows for further processing comprises providing the summary information to a report generation process.

9. The method of claim 1, further comprising querying, by the processor of the medical records processing system, the LLM for a summary of the summary information.

10. A system comprising:
a processor; and
a memory coupled with and readable by the processor and storing therein a set of instructions which, when executed by the processor, causes the processor to:
receive a set of documents comprising medical records;
pre-process the received set of documents into a set of medical information;
query a Large Language Model (LLM) for summary information of the set of relevant medical information; and
provide the summary information to one or more workflows for further processing.

11. The system of claim 10, wherein pre-processing the received set of documents into a set of medical information comprises sorting and classifying the received set of documents.

12. The system of claim 10, wherein pre-processing the received set of documents into a set of medical information comprises running an Optical Character Recognition (OCR) process on the received set of documents.

13. The system of claim 10, wherein querying the LLM for summary information of the set of medical information comprises providing, to the LLM, a sequence of successively more specific queries related to the set of medical information and summary information returned by the LLM.

14. The system of claim 10, wherein providing the summary information to one or more workflows for further processing comprises providing the summary information to a review and processing tool and wherein the review and processing tool provides a user interface through which a user interacts with the summary information.

15. The system of claim 14, wherein the user interface provided by the review and processing tool presents the summary information organized into a plurality of groups and provides an indication of specific information within the summary information requiring review.

16. The system of claim 14, wherein the set of instructions further causes the processor to:
receive a modification of the summary information from the review and processing tool based on an input from the user through the user interface; and
update the summary information based on the received modification.

17. The system of claim 10, wherein providing the summary information to one or more workflows for further processing comprises providing the summary information to a report generation process.

18. The system of claim 10, wherein the set of instructions further causes the processor to query the LLM for a summary of the summary information.

19. A non-transitory, computer-readable medium comprising a set of instructions stored therein which, when executed by a processor, causes the processor to:
receive a set of documents comprising medical records;
pre-process the received set of documents into a set of medical information;
query a Large Language Model (LLM) for summary information of the set of relevant medical information; and
provide the summary information to one or more workflows for further processing.

20. The non-transitory, computer-readable medium of claim 19, wherein pre-processing the received set of documents into a set of medical information comprises sorting and classifying the received set of documents and running an Optical Character Recognition (OCR) process on the received set of documents.

21. The non-transitory, computer-readable medium of claim 19, wherein querying the LLM for summary information of the set of medical information comprises providing, to the LLM, a sequence of successively more specific queries related to the set of medical information and summary information returned by the LLM.

22. The non-transitory, computer-readable medium of claim 19, wherein the set of instructions further causes the processor to query the LLM for a summary of the summary information.
